(19) United States
(12) Patent Application Publication
(10) Pub. No.: US 2018/0015196 A1
(43) Pub. Date: Jan. 18, 2018

(54) ATTACHABLE AND MOVABLE DEODORIZER FOR ENCLOSED SPACE

(71) Applicant: CONARY ENTERPRISE CO., LTD, Taipei City (TW)

(72) Inventors: SHIH JUNG DANICA HUANG, TAIPEI CITY (TW); CHIH-LIANG LIN, TAIPEI CITY (TW)

(21) Appl. No.: 15/715,387

(22) Filed: Sep. 26, 2017

Related U.S. Application Data
(63) Continuation-in-part of application No. 14/947,097, filed on Nov. 20, 2015.

(51) Int. Cl. A61L 9/20; B01D 53/88

(52) U.S. Cl. CPC A61L 9/205; B01D 53/88; B01D 2258/06; B01D 2257/91; A61L 2209/14; B01D 2255/802; B01D 2259/804; B01D 2257/90

(57) ABSTRACT

An attachable and movable deodorizer for enclosed space has a body with a plurality of inlet holes and outlet holes. The body has a fan, a photocatalyst filter, and an ultraviolet (UV) control circuit board connected to an UV-A LED lamp. Whereby the body is able to be disposed or attached in an enclosed space such as trash bins and shoe cabinets and to suck in air in the enclosed space by the fan for the air to pass through the photocatalyst filter and then to be discharged from the outlet holes. With the photocatalyst filter illuminated by the UV-A LED lamp, the stench and dusts in the air is therefore filtered and deodorized in the photocatalysis process.

This patent application is a continuation-in-part of Ser. No. 14/947,097 filed on Nov. 20, 2015, currently pending.

### BACKGROUND OF THE INVENTION

### 1. Field of the Invention

The present invention relates to a deodorizer, particularly to one that can be attached within and removed from an enclosed space and has an ultraviolet-A (UV-A) LED lamp to illuminate a photocatalyst filter for photocatalysis and therefore deodorize a stench and filter the air in the enclosed space.

### 2. Description of the Related Art

In spaces with poor ventilation, there are often bacteria, dusts, and suspended substance in the air; in some enclosed spaces such as shoe cabinets and trash bins, it is even worse—thriving bacteria and stenches spreading all over. An ultraviolet-C (UV-C) has a wavelength between 200-280 nm. It can directly destroy the DNA structure of bacteria, virus, and microorganism to stop them from thriving and inhabitation. However, when it comes to shoe cabinets, UV-C can only deal with the bacteria but can do nothing about the stench, and the UV-C can cause serial damages to human body if exposed.

Photocatalyst is developed from UV lighting; it is a series of chemical reaction as the energy of lights absorbed for the catalysis process with titanium dioxide (TiO₂) nanoparticles being the catalyst. The photocatalytic activity depends on the ability of the catalyst to create electrons and electron-holes, which generate hydroxyl radicals OH⁻ to decompose the organic molecules, so as to easily turn the stench chemical substance in the air into clean and harmless substance. As a result, photocatalyst is able to deodorize, prevent bacteria and mildew from thriving, and decompose dirt. When trashes are put inside a trash bin as in an enclosed space, the bacteria would rapidly thrive due to poor air circulation and therefore produce a stench. With an LED lamp disposed inside the trash bin and a photocatalyst is spread on an inner surface of a lid of the trash bin, the air near the photocatalyst can be decomposed and deodorized, but deep down the trash bin, the air far from the photocatalyst cannot be processed since spreading photocatalyst on the inner surface of the lid can only deal with partial air in the trash bin.

To sum up, UV-C lamps cannot fully deodorize the air within enclosed space of shoe cabinets, and photocatalyst spread on a lid of trash bins with a LED lamp installed therein cannot entirely deodorize the air filled within enclosed space of trash bins either. Hence, it is desirable to provide a device that is conveniently installed and that completely deodorizes and filter the air within an enclosed space without potential damages to human body.

A photo-catalytic oxidizing assembly is disclosed in US publication No. 2013/0052090. The assembly mainly comprises a housing including an outer top housing, a latch assembly, an inner top housing, a front panel, a rear panel, an air inlet grill, an air outlet grill, a bottom inner housing assembly, an outer bottom assembly, and a cord wrap cleat; the air inlet grill further has an air inlets and the air outlet grill further has an air outlet. The assembly is therefore a complex device with a heavy weight, making it difficult to be moved or attached as required. A space is formed when the elements above are assembled; the space further has a pre-filter, an electrostatic precipitator cell, a photo-catalytic oxidizing assembly, and a fan assembly. Airflow enters via the air inlet through the photo-catalytic oxidizing assembly and the fan assembly, and exists from the air outlet, forming a single direction of the airflow, thereby confining the deodorization function.

### SUMMARY OF THE INVENTION

A primary objective of the present invention is to provide an attachable deodorizer for enclosed space to deodorize and filter the air in the enclosed space by a photocatalyst filter.

Another objective of the present invention is to provide an attachable and removable deodorizer for an enclosed space such as space within trash bins and shoe cabinets.

Yet another objective of the present invention is to provide a deodorizer disposed in an enclosed space that is able to instantly cease the operation of deodorization and filtering when the space becomes opened, preventing potential damages to human body.

In order to achieve the object above, the present invention comprises a body having a placing space therein, a first part and a second part, wherein the first and second parts of the body are arranged in corresponding shapes for engagement; the first part has a first depressed surface at a front end thereof and a lid section detachably engaged the first depressed surface to form an inlet space, said first depressed surface having a protruding section at a center thereof and a plurality of ventilation openings surrounding said protruding section, and said lid section including a plurality of inlet holes; and the second part has a plurality of outlet holes arranged on a side thereof and a second depressed surface at a read end thereof, said second depressed surface further engaged with an attaching piece for the body to be attached to an object;

a fan disposed in the placing space adjacent to the inlet holes, wherein the fan is an axial flow fan and has a plurality of engaging holes, and a rear end of the fan is facing towards the first depressed surface;

a photocatalyst filter disposed at a front of the fan for air sucked in by the fan to pass through the photocatalyst filter, wherein the front of the fan is facing towards the photocatalyst filter and a supporting element is disposed in-between the fan and the photocatalyst filter, said supporting element including a plurality of guiding openings, a frame to support the photocatalyst filter and a plurality of supporting rods engaged through the engaging holes of the fan for positioning the fan and the photocatalyst filter, thereby forming a space with a height for ventilation in directions according to arrangement of the inlet holes and outlet holes;

an ultraviolet control circuit board including an ultraviolet-A LED lamp, a microcontroller and a power controlling chip, said ultraviolet control circuit board disposed in the second part of the body and electrically connected to the fan for the ultraviolet-A LED lamp to illuminate and catalyze the photocatalyst filter for deodorization; and

a wire connected to the ultraviolet control circuit board with an end for external electricity supply flowing therethrough for the ultraviolet control circuit board and the fan to operate;

whereby when the body is attached in an enclosed space, dusts and suspended substances filled within the enclosed space would be sucked in by the fan to the body via the inlet holes, pass through the photocatalyst filter and then be discharged from the body via the outlet holes, and the photocatalyst filter is illuminated by the ultraviolet-A LED lamp to deodorize a stench and filter chemical substances within the enclosed space.

Furthermore, the attaching piece is a magnet, a touch fastener or a double-sided adhesive for the body to be attached onto and removed from the enclosed space. The power controlling chip is set a time for activation and a time for suspension for the fan and the ultraviolet-A LED lamp to form a cycle of operation.

The second part of the body further includes a sensor engaged thereon and electrically connected to the microcontroller of the ultraviolet control circuit board for detecting opening or closing of the opening section so that when the opening section is opened, the power controlling chip is able to stop operation of the fan and the ultraviolet-A LED lamp, and when the opening section is closed, the power controlling chip is able to reactivate operation of the fan and the ultraviolet-A LED lamp. The second part of the body further has a manual switch disposed thereon and electrically connected to the microcontroller of the ultraviolet control circuit board so that when the stench in the enclosed space is too strong, the manual switch can be pressed to reset the cycle of operation of the fan and the ultraviolet-A LED lamp and reactivate the deodorizer.

The wire has the other end thereof connected to a battery device installed with rechargeable or non-rechargeable batteries therein. And the ultraviolet-A LED lamp has a wavelength between 360-400 nm, preferably 380 nm.

With structures disclosed above, the present invention has the fan to suck in air from the enclosed space to the body and then deodorize the stench and filter the air by having the photocatalyst filter illuminated and catalyzed by the UV-A LED lamp. Also, the present invention has the attaching piece made of magnets, touch fasteners, or double-sided adhesive for the deodorizer to be easily attached and removed within the enclosed space. In addition, the sensor is electrically connected to the microcontroller for detection of the opening section so that the power controlling chip is able to instantly cease operations of the fan and the UV-A LED lamp upon the space becoming opened, preventing potential damages to human body.

### BRIEF DESCRIPTION OF THE DRAWINGS

FIG. 1 is a perspective view of the present invention;

FIG. 2 is another perspective view of the present invention;

FIG. 3 is an exploded view of the present invention;

FIG. 4 is a schematic diagram illustrating directions of air flowing through the present invention;

FIG. 5 is a sectional view of the present invention, showing a space with a height formed within the present invention for ventilation;

FIG. 6 is a circuit diagram of the present invention;

FIG. 7A is a schematic diagram of the present invention applied to a shoe cabinet;

FIG. 7B is another schematic diagram of the present invention applied to the shoe cabinet;

FIG. 8A is a schematic diagram of the present invention applied to a trash bin;

FIG. 8B is another schematic diagram of the present invention applied to the trash bin; and

FIG. 9 is a schematic diagram illustrating the present invention held by a human hand.

### DETAILED DESCRIPTION OF THE PREFERRED EMBODIMENT

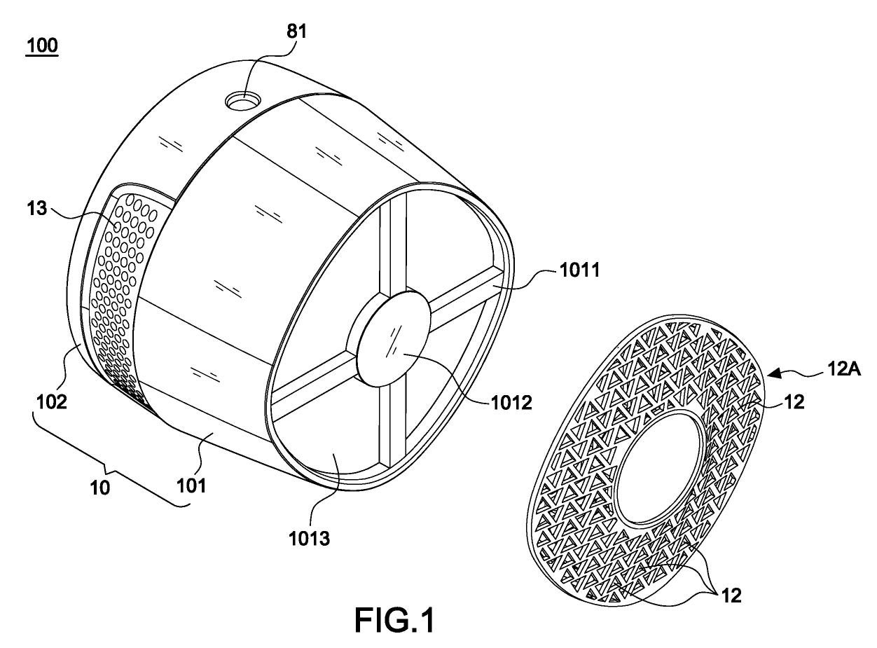

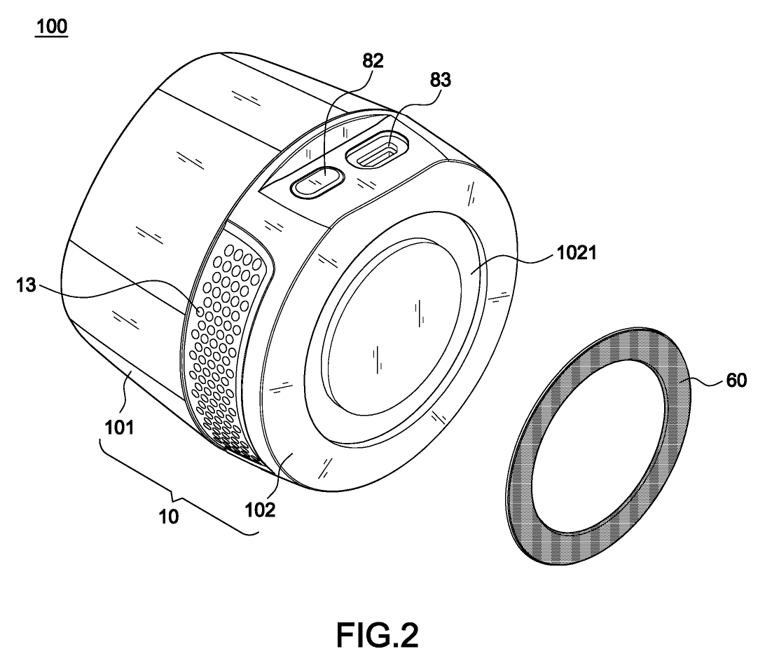

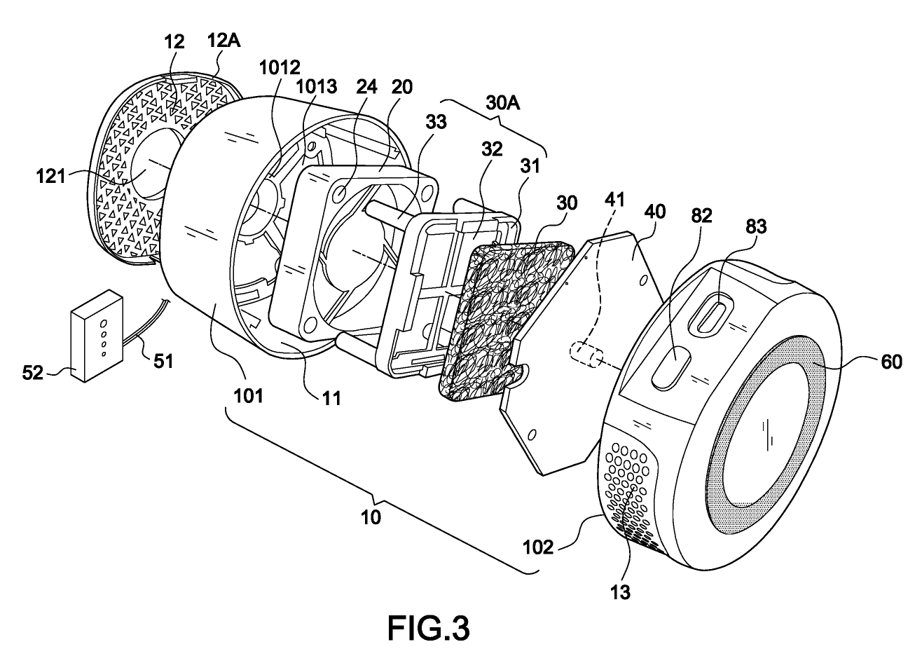

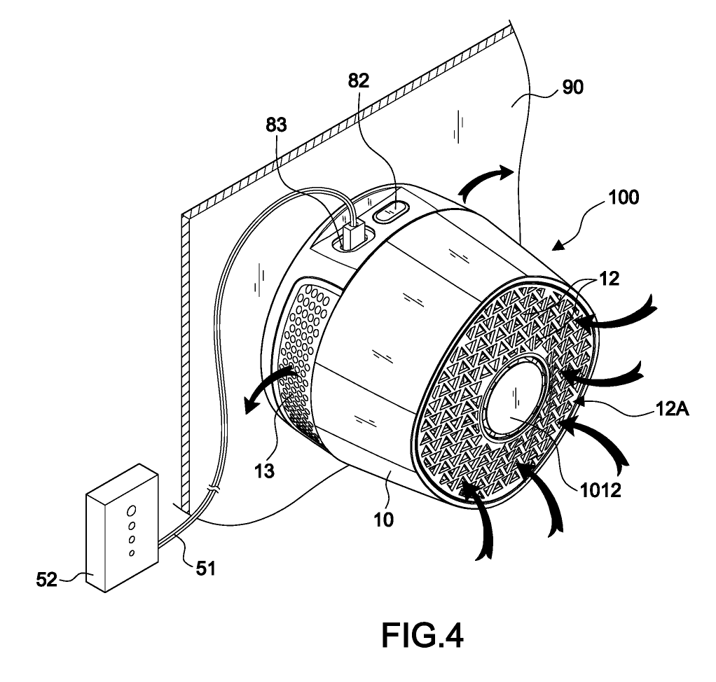

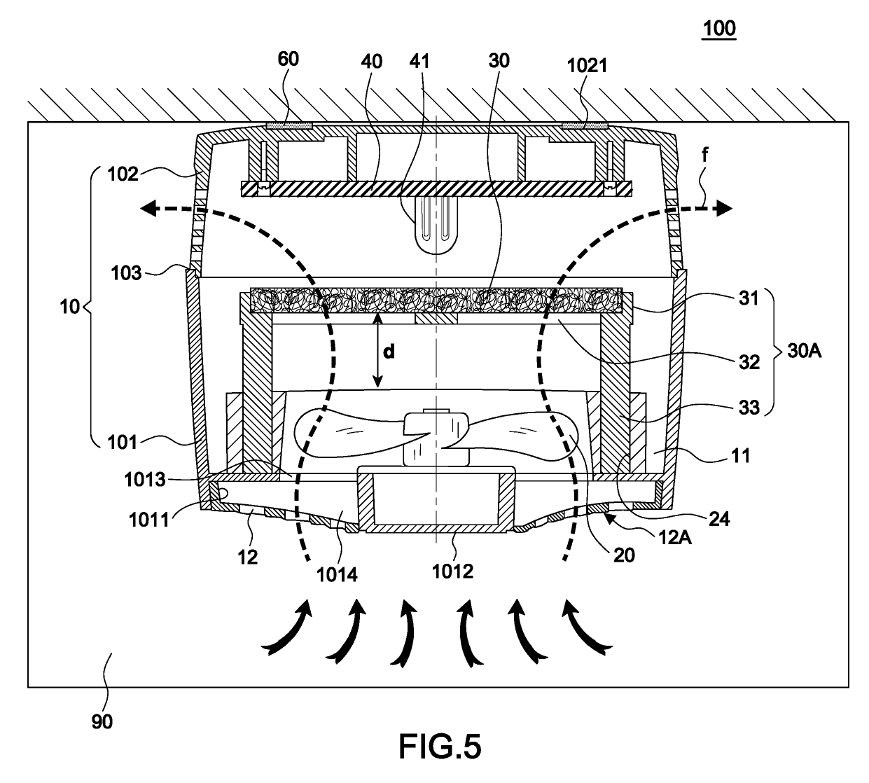

Referring to FIGS. 1-9, in a preferred embodiment, the attachable and movable deodorizer 100 mainly includes a body 10, a fan 20, a photocatalyst filter 30, an ultraviolet (UV) control circuit board 40, and a wire 51.

The body 10 has a placing space 11 therein, a first part 101 and a second part 102; the first and second parts 101, 102 are arranged in corresponding shapes for engagement. The first part 101 has a first depressed surface 1011 at a front end thereof and a lid section 12A detachably engaged the first depressed surface 1011 to form an inlet space 1014. The first depressed surface 1011 has a protruding section 1012 at a center thereof and a plurality of ventilation openings 1013 surrounding the protruding section 1012, and the lid section 12A includes a plurality of inlet holes 12. The second part 102 has a plurality of outlet holes 13 arranged on a side thereof and a second depressed surface 1021 at a read end thereof. The second depressed surface 1021 is further engaged with an attaching piece 60 for the body 10 to be attached to an object. In this embodiment, the attaching piece 60 is a magnet, a touch fastener or a double-sided adhesive for the body 10 to be attached in and removed from an enclosed space 90 such as space in a trash bin 300 or a shoe cabinet 200.

The fan 20 is disposed in the placing space 11 adjacent to the inlet holes 12; it is an axial flow fan and has a plurality of engaging holes 24, and a rear end of the fan 20 is facing towards the first depressed surface 1011.

The photocatalyst filter 30 is disposed at a front of the fan 20 for air sucked in by the fan 20 to pass through the photocatalyst filter 30. The front of the fan 20 is facing towards the photocatalyst filter 30 and a supporting element 30A is disposed in-between the fan 20 and the photocatalyst filter 30. The supporting element 30A includes a plurality of guiding openings 32, a frame 31 to support the photocatalyst filter 30 and a plurality of supporting rods 33 engaged through the engaging holes 24 of the fan 20 for positioning the fan 20 and the photocatalyst filter 30, thereby forming a space with a height d for ventilation in different directions f according to arrangement of the inlet holes 12 and outlet holes 13.

The UV control circuit board 40 includes an ultraviolet-A LED lamp 41, a microcontroller 42 and a power controlling chip 43. The UV control circuit board 40 is disposed in the second part 102 of the body 10 and is electrically connected to the fan 20 for the ultraviolet-A LED lamp 41 to illuminate and catalyze the photocatalyst filter 30 for deodorization. The power controlling chip 43 is set a time for activation T1 and a time for suspension T2 for the fan 20 and the ultraviolet-A LED lamp 41 to form a cycle of operation.

The wire 51 is connected to the UV control circuit board 40 with an end for external electricity supply flowing therethrough for the UV control circuit board 40 and the fan 20 to operate. In this embodiment, the second part 102 of the body 10 has a USB port 83 arranged thereon for electrical connection to another end of the wire 51.

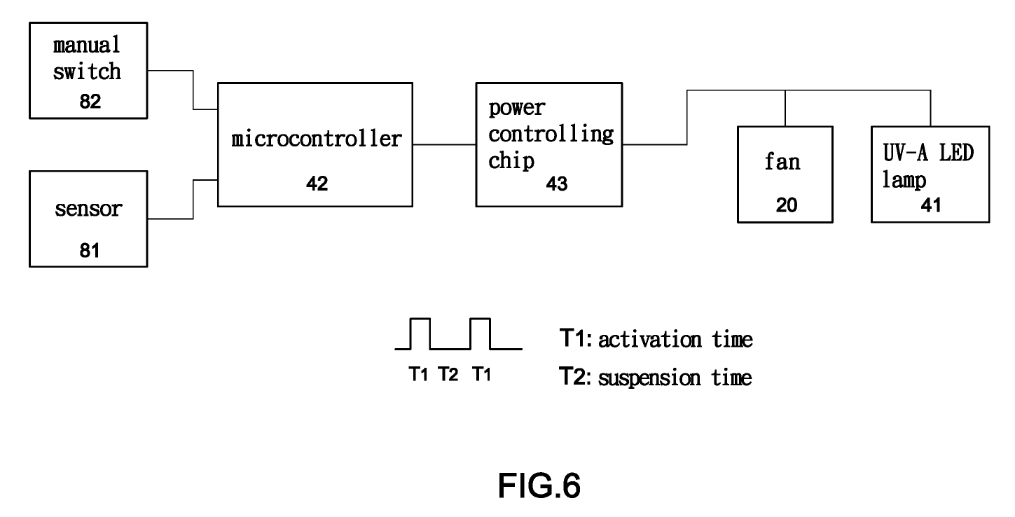

In addition, the second part 102 of the body 10 further includes a sensor 81 and a manual switch 82, both arranged on the second part 102 and electrically connected to the microcontroller 42 of the UV control circuit board 40 as shown in FIG. 6. The sensor 81 detects opening or closing of an opening section so that when the opening section is opened, the power controlling chip 43 is able to stop operation of the fan 20 and the ultraviolet-A LED lamp 41, and when the opening section is closed, the power controlling chip 43 is able to reactivate operation of the fan 20 and the ultraviolet-A LED lamp 41. When the stench in the enclosed space 90 is too strong, the manual switch 82 can be pressed to reset the cycle of operation of the fan 20 and the ultraviolet-A LED lamp 41 and reactivate the deodorizer 100. In this embodiment, the wire 51 has the other end thereof connected to a battery device 52 installed with rechargeable or non-rechargeable batteries therein.

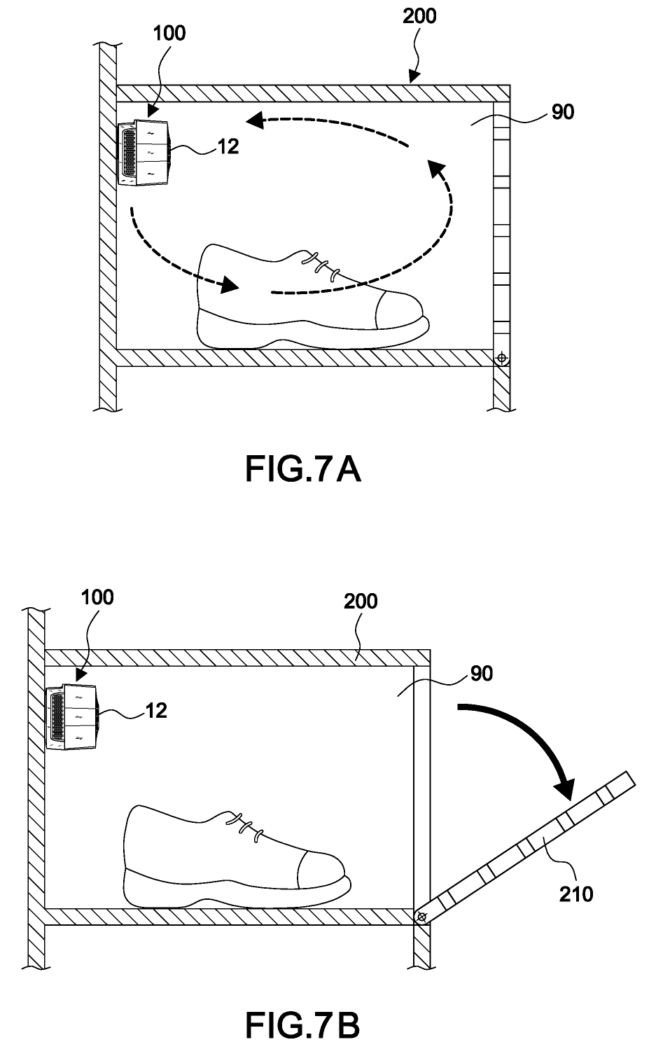

Referring to FIGS. 7A, the deodorizer 100 is attached on a wall of a shoe cabinet 200 by the attaching piece 60 and a shoe is disposed therein. Since it is an enclosed space 90, the bacteria in the shoe would grow and even produce a stench within the enclosed space 90 and dusts and suspended substances are kept in the enclosed space 90 as well. With operation of the deodorizer 100, the dust, suspended substances and the bacteria in the enclosed space 90 are sucked into the body 10 by the fan 20 via the inlet holes 12, filtered by the photocatalyst filter 30 and discharged from the outlet holes 13. The photocatalyst filter 30 is able to filter the substances and the stench by illumination of the ultraviolet-A LED lamp 41. Therefore, the air within the enclosed space 90 of the shoe cabinet 200 becomes clean. FIG. 7B illustrates a door 210 of the shoe cabinet 200 functions as the opening section. When the enclosed space 90 is detected opened by the sensor 81, the microcontroller 42 would send out signals to the power controlling chip 43 to cease operations of the ultraviolet-A LED lamp 41 and the fan 20, thereby preventing from potential damages of ultraviolet exposure.

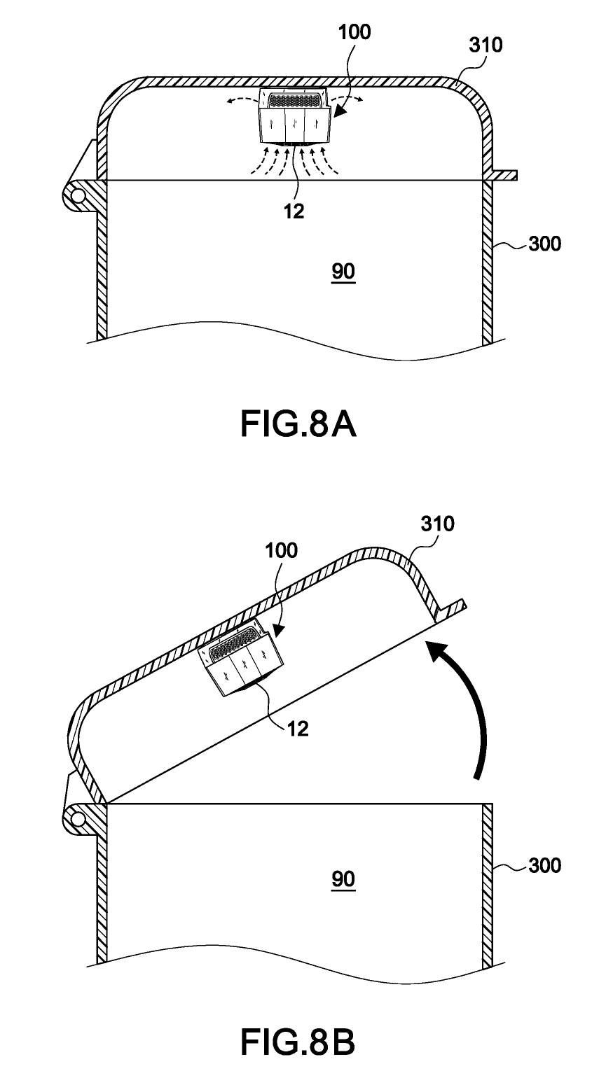

Referring to FIGS. 8A, the deodorizer 100 is attached under a lid 310 of a trash bin 300 by the attaching piece 60. Since it is an enclosed space 90, the air within the trash bin 300 is also full with bacteria, dusts and suspended substances. The stench in the enclosed space 90 might be even worse. With operation of the deodorizer 100, the dust, suspended substances and the bacteria in the enclosed space 90 are sucked into the body 10 by the fan 20 via the inlet holes 12, filtered by the photocatalyst filter 30 and discharged from the outlet holes 13. The photocatalyst filter 30 is able to filter the substances and the stench by illumination of the ultraviolet-A LED lamp 41. Therefore, the air within the enclosed space 90 of the trash bin 300 becomes clean. FIG. 8B illustrates the lid 310 functioning as the opening section is opened. With the sensor 81, the microcontroller 42 would send out signals to the power controlling chip 43 to cease operations of the ultraviolet-A LED lamp 41 and the fan 20, thereby preventing from potential damages of ultraviolet exposure.

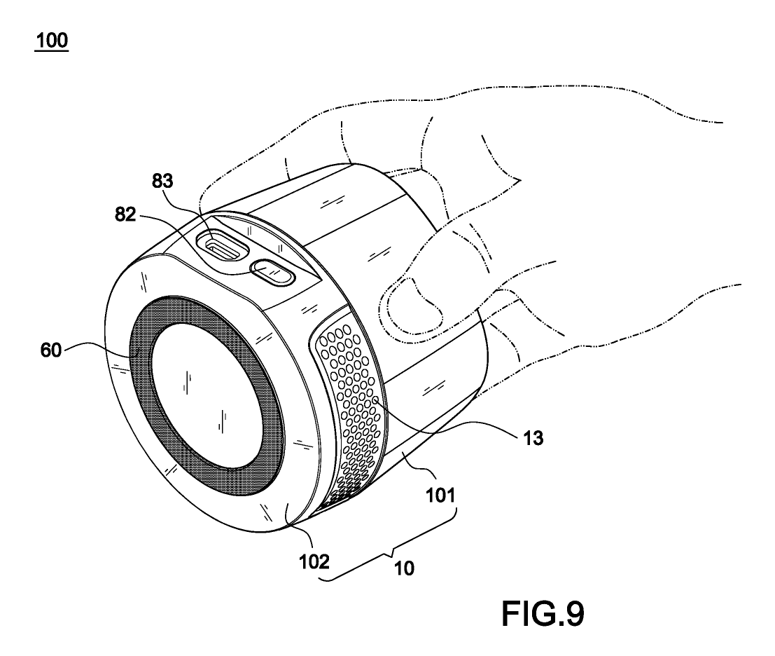

As shown in FIG. 9, the deodorizer 100 of the present invention has a small volume and light weight that can be held by a single hand and moved easily. In addition, the present invention has features concluded as following.

1. The deodorizer 100 is able to filter the air within the enclosed space 90 by the photocatalyst filter 30.

2. The deodorizer 100 can be easily attached onto and removed from the enclosed space 90.

3. The deodorizer 100 can instantly cease the filtering operation when detecting the opening section is opened, thereby preventing from potential damages of ultraviolet exposure.

On the other hand, the present invention is further analyzed and compared with the invention disclosed in US Publication No. 2013/0052090.

| | Invention | |
|---|---|---|
| | | Photo-catalytic oxidizing |
| | Attachable and movable | assembly (disclosed in US |
| | deodorizer (the present | Publication No. |
| Comparison | invention) | 2013/0052090) |
| Volume | Simple engagement of the | A housing of the assembly |
| | first and second parts allows | includes an outer top |
| | the deodorizer to have a | housing, a latch assembly, |
| | small volume and to be | an inner top housing, a front |
| | easily movable; | panel, a rear panel, an air |
| | furthermore, with the | inlet grill, an air outlet grill, |
| | attaching piece, the | a bottom inner housing |
| | deodorizer can be attached | assembly, an outer bottom |
| | to an object or to a place. | assembly, and a cord wrap |
| | | cleat, making it a complex |
| | | device with a heavy weight. |
| Airflow | The supporting element | In the housing, the device |
| | disposed in-between the fan | includes a pre-filter, an |
| | and the photocatalyst filter | electrostatic precipitator |
| | forms the space with the | cell, a photo-catalytic |
| | height for ventilation in | oxidizing assembly, and a |
| | different directions | fan assembly. Airflow enters |
| | according to arrangement of | via the air inlet through the |
| | the inlet holes and outlet | photo-catalytic oxidizing |
| | holes; in other words, the | assembly and the fan |
| | deodorizer can filter and | assembly, and exists from |
| | deodorize the air without | the air outlet, forming a |
| | additional filtering element. | single direction of the |
| | | airflow. Besides, the device |
| | | cannot enhance |
| | | deodorization. |

In short, the present invention has a small volume and light weight that can be attached and moved as needed, and it enables airflow in different directions in operation; while the assembly disclosed in US 2013/0052090 has a great volume and heavy weight and only enables airflow in single direction, making it difficult for movement and attachment if needed and the deodorization function is therefore limited.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. An attachable and movable deodorizer for enclosed space comprising:
a body having a placing space therein, a first part and a second part, wherein the first and second parts of the body are arranged in corresponding shapes for engagement; the first part has a first depressed surface at a front end thereof and a lid section detachably engaged the first depressed surface to form an inlet space, said first depressed surface having a protruding section at a center thereof and a plurality of ventilation openings surrounding said protruding section, and said lid section including a plurality of inlet holes; and the second part has a plurality of outlet holes arranged on a side thereof and a second depressed surface at a read end thereof, said second depressed surface further engaged with an attaching piece for the body to be attached to an object;
a fan disposed in the placing space adjacent to the inlet holes, wherein the fan is an axial flow fan and has a plurality of engaging holes, and a rear end of the fan is facing towards the first depressed surface;
a photocatalyst filter disposed at a front of the fan for air sucked in by the fan to pass through the photocatalyst filter, wherein the front of the fan is facing towards the photocatalyst filter and a supporting element is disposed in-between the fan and the photocatalyst filter, said supporting element including a plurality of guiding openings, a frame to support the photocatalyst filter and a plurality of supporting rods engaged through the engaging holes of the fan for positioning the fan and the photocatalyst filter, thereby forming a space with a height for ventilation in directions according to arrangement of the inlet holes and outlet holes;
an ultraviolet control circuit board including an ultraviolet-A LED lamp, a microcontroller and a power controlling chip, said ultraviolet control circuit board disposed in the second part of the body and electrically connected to the fan for the ultraviolet-A LED lamp to illuminate and catalyze the photocatalyst filter for deodorization; and
a wire connected to the ultraviolet control circuit board with an end for external electricity supply flowing therethrough for the ultraviolet control circuit board and the fan to operate;
whereby when the body is attached in an enclosed space, dusts and suspended substances filled within the enclosed space would be sucked in by the fan to the body via the inlet holes, pass through the photocatalyst filter and then be discharged from the body via the outlet holes, and the photocatalyst filter is illuminated by the ultraviolet-A LED lamp to deodorize a stench and filter chemical substances within the enclosed space.

2. The attachable and movable deodorizer for enclosed space as claimed in claim 1, wherein the attaching piece is a magnet, a touch fastener or a double-sided adhesive for the body to be attached in and removed from the enclosed space.

3. The attachable and movable deodorizer for enclosed space as claimed in claim 1, wherein the power controlling chip is set a time for activation and a time for suspension for the fan and the ultraviolet-A LED lamp to form a cycle of operation.

4. The attachable and movable deodorizer for enclosed space as claimed in claim 1, wherein the second part of the body further includes a sensor engaged thereon and electrically connected to the microcontroller of the ultraviolet control circuit board for detecting opening or closing of the opening section so that when the opening section is opened, the power controlling chip is able to stop operation of the fan and the ultraviolet-A LED lamp, and when the opening section is closed, the power controlling chip is able to reactivate operation of the fan and the ultraviolet-A LED lamp.

5. The attachable and movable deodorizer for enclosed space as claimed in claim 4, wherein the second part of the body further includes a manual switch arranged thereon and electrically connected to the microcontroller of the ultraviolet control circuit board so that when the stench in the enclosed space is too strong, the manual switch can be pressed to reset the cycle of operation of the fan and the ultraviolet-A LED lamp and reactivate the deodorizer.

6. The attachable and movable deodorizer for enclosed space as claimed in claim 1, wherein the wire has the other end thereof connected to a battery device installed with rechargeable or non-rechargeable batteries therein.

7. The attachable and movable deodorizer for enclosed space as claimed in claim 1, wherein the ultraviolet-A LED lamp has a wavelength between 360-400 nm.

8. The attachable and movable deodorizer for enclosed space as claimed in claim 7, wherein the wavelength of the ultraviolet-A LED lamp is 380 nm.
